(19) United States
(12) Patent Application Publication
(10) Pub. No.: US 2025/0255726 A1
(43) Pub. Date: Aug. 14, 2025

(54) VARIABLE GEOMETRY RADIAL HEAD STEM

(71) Applicant: Acumed LLC, Hillsboro, OR (US)

(72) Inventors: Michael Zwolinski, Willoughby, OH (US); Larry Ehmke, Portland, OR (US); Jeffrey Sander, Parkville, MO (US)

(21) Appl. No.: 19/051,502

(22) Filed: Feb. 12, 2025

Related U.S. Application Data
(60) Provisional application No. 63/552,518, filed on Feb. 12, 2024.

(51) Int. Cl. A61F 2/38

(52) U.S. Cl. CPC A61F 2/3804; A61F 2002/3827

(57) ABSTRACT

Provided herein are prostheses for replacement of a proximal end of a radius bone. The prosthesis may include a head and a stem directly or indirectly coupled to the head. The stem may be configured to fit in a bore of a radius bone. The stem may define a virtual cylindrical shaft having a central axis. The stem may include a side surface portion coinciding with a side surface portion of the virtual cylindrical shaft. The virtual cylindrical shaft may include a portion occupied by the stem and a vacant portion.

### CROSS-REFERENCE TO RELATED APPLICATIONS

The present disclosure claims priority to U.S. Provisional Patent Application 63/552,518 titled “VARIABLE GEOMETRY RADIAL HEAD STEM”, which was filed on Feb. 12, 2024, and which is incorporated herein in its entirety.

### BACKGROUND

The head of a bone generally includes a rounded extension that is used to form a part of a joint. The head is the main articulating surface with the adjacent bone. When the head of a bone becomes fractured or otherwise damaged, it may be difficult to repair. When the head is irreparable, prosthetic replacement of the head of the bone may be necessary to keep the joint intact.

Specifically, prosthetic replacement of the radial head in the elbow joint may be performed. The elbow joint is comprised of the humerus, the radius, and the ulna. The radial head articulates at its end with the capitellum of the humerus, to form the humeroradial joint, and on its side with the radial notch of the ulna, to form the proximal radioulnar joint.

A radial head replacement may include removing the entirety of the radial head and replacing it with a radial head prosthesis. Typical radial head replacements include a stem portion and a head portion. The stem portion is used to secure the head to the radius, while the head is configured to mimic the articulating surfaces of the radial head. During replacement, a bore may be drilled into the radius bone. The stem portion of the radial head prosthesis may be configured to fit in the bore of the radius to secure the prosthesis to the radius.

Upon implantation of the radial head prosthesis, it may be difficult to maneuver the stem into a bore of the radius without interfering with the capitellum and surrounding soft tissue. This may result in the surgeon having to remove more bone of the radius than necessary to fit the radial head prosthesis into place or resect additional soft tissue. There exists a need for a radial head prosthesis that allows ease of insertion.

### SUMMARY

The present disclosure provides a new and innovative radial head prostheses that may be easier to insert than current solutions. The prostheses provided herein may allow a surgeon to implant a radial head prosthesis without having to remove bone from the radius unnecessarily or resect additional soft tissue. The prostheses according to the present disclosure may include a monoblock design where the head is directly coupled to the stem, or the prostheses may include a modular design where the head is indirectly coupled to the stem, such as through use of a morse taper connection.

One aspect of the present disclosure provides a radial head prosthesis including a stem defining a virtual cylindrical shaft having a central axis. The stem may include a side surface portion coinciding with a side surface portion of the virtual cylindrical shaft.

Further, the virtual cylindrical shaft may include a portion occupied by the stem and a vacant portion. The stem may further define a plane perpendicular to the central axis of the virtual cylindrical shaft, a first axis on the plane and passing through the central axis of the virtual cylindrical shaft, and a second axis on the plane, passing through the central axis of the virtual cylindrical shaft, and perpendicular to the first axis.

When viewed from the first axis, the virtual cylindrical shaft may be divided, by the central axis, into a first portion and a second portion. Over 80% of the first and second portions may be covered by the stem. When viewed from the second axis, the virtual cylindrical shaft may be divided, by the central axis, into a third portion and a fourth portion. Over 95% of the third portion may be covered by the stem and less than 90% of the fourth portion may be covered by the stem.

In some examples, a volume ratio between the portion occupied by the stem and the vacant portion in the virtual cylindrical shaft may be in a range of about 1:1.5 to about 7:1. In other examples, a volume ratio between the portion occupied by the stem and the vacant portion in the virtual cylindrical shaft may be in a range of about 1:1.5 to about 1.5:1.

Additional features and advantages of the disclosed methods are described in, and will be apparent from, the following Detailed Description and the Figures.

### BRIEF DESCRIPTION OF THE FIGURES

FIGS. 1A and 1B illustrates a perspective view of a prosthesis for replacement of a proximal end of a radius bone according to an example of the present disclosure.

FIGS. 2A and 2B illustrate the prosthesis of FIG. 1A from a first axis (FIG. 2A) and from a second axis (FIG. 2B).

FIG. 3 illustrates the prosthesis of FIG. 1A from a first axis.

FIG. 4 illustrates a cross-sectional view taken along the line 4-4 of FIG. 2A.

FIG. 5 illustrates a second end portion of the prosthesis of FIG. 1A according to an example of the present disclosure.

FIG. 6 illustrates a perspective view of a prosthesis for replacement of a proximal end of a radius bone according to an example of the present disclosure.

FIGS. 7A and 7B illustrate the prosthesis of FIG. 6 from a first axis (FIG. 7A) and from a second axis (FIG. 7B).

FIG. 8 illustrates the prosthesis of FIG. 6 from the first axis.

FIG. 9 illustrates a cross-sectional view taken along the line 9-9 of FIG. 7A.

FIG. 10 illustrates a second end portion of the prosthesis of FIG. 6 according to an example of the present disclosure.

FIGS. 11-14 illustrate modified prostheses of FIG. 6 from the second axis.

FIG. 15 illustrates a graph showing the ease of insertion of various prostheses according to examples of the present disclosure.

FIG. 16 illustrates a relative measurement of ease of insertion according to an example of the present disclosure.

FIGS. 17A-17J illustrate alternative geometries of prostheses according to examples of the present disclosure.

FIGS. 18A-18F illustrate alternative geometries of prostheses according to examples of the present disclosure.

### DETAILED DESCRIPTION OF EXAMPLES

The present disclosure is directed to a prosthesis for a proximal end of a radius bone that allows ease of insertion into a bore of the radius bone.

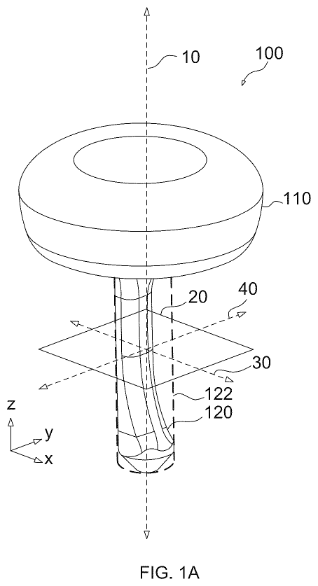

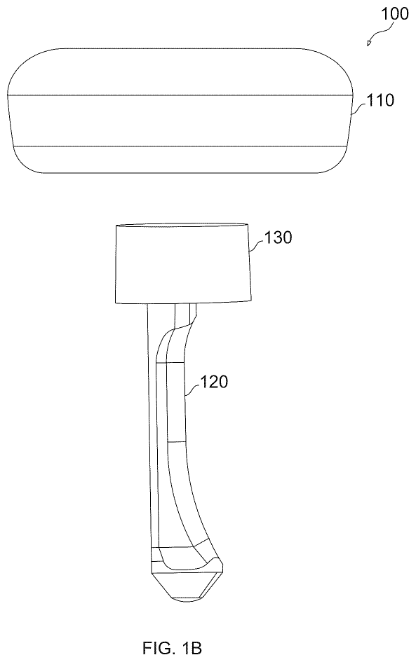

FIGS. 1A to 5 illustrate an example prosthesis 100 for replacement of a proximal end of a radius bone according to an example of the present disclosure. Referring to FIGS. 1A and 1B, the prosthesis 100 may include a head 110 and a stem 120. The stem 120 may be directly or indirectly coupled to the head 110. In some examples, the head 110 may have a central axis 10. In some examples, the central axis 10 of the head 110 may coincide with a central axis 10 of the stem 120. In some examples, when coupled to the stem 120, the head 110 may be orthogonal to the stem 120. In other examples, when coupled to the stem 120, the head 110 may be tilted relative to a central axis 10 of the stem 120.

As shown in FIG. 1A, in some examples, the prosthesis 100 may have a monoblock design where the stem 120 may be directly coupled to or formed integrally with a bottom surface of the head 110. As shown in FIG. 1B, in other examples, the prosthesis 100 may have a modular design where the stem 120 may be indirectly coupled to the head 110, such as through a morse taper connection. The stem 120 may be directly coupled to a trunnion 130, and the head 110 may have an opening configured to receive the trunnion 130. This may allow the head 110 to be removably coupled to the stem 120 and/or the trunnion 130.

The head 110 may be configured to mimic the head of a radius bone. For example, the head 110 may be (substantially) cylindrical in shape and may include a top surface configured to engage with a capitellum of the humerus bone to form the elbow joint. Additionally or alternatively, the head 110 may include a side surface configured to engage with a radial notch of the ulna.

The head 110 and/or stem 120 may be comprised of any suitable material, such as biocompatible materials (metal alloys (titanium alloys, cobalt chromium alloys, stainless steel, etc.), composite materials, plastics (polyethylene, among others), ceramics, and/or the like), and/or bioabsorbable materials (polygalactic acid (PGA), polylactic acid (PLA), copolymers thereof, etc.), among others. In some examples, the stem 120 may be made of a substantially rigid material so that the prosthesis is secured in the bore of the radius bone without substantial movement.

The stem 120 may include an elongated shaft configured to be received in a bore of the radius bone to secure the prosthesis 100 to the radius. In some examples, the stem 120 may be (substantially) cylindrical or have any desired shape. For example, as can be seen in FIGS. 1A, the stem 120 may define a virtual cylinder 122 with portions occupied by the stem 120 and portions removed from the virtual cylinder 122, also known as vacant portions. In some examples, the virtual cylinder 122 may have a diameter between about 6 mm and about 12 mm, for example, 6 mm, 7 mm, 8 mm, 9 mm, 10 mm, 11 mm, or 12 mm. In other examples, the virtual cylinder 122 may have any other suitable diameter. In some examples, a side surface portion of the stem 120 (for example, at least one point of the stem 120) may coincide with a side surface portion of the virtual cylinder 122. Removing portions of the virtual cylinder 122 from the stem 120 (e.g., the vacant portions of the stem 120) may allow better insertion angles for implanting the prosthesis 100 into the bore of the radius.

The virtual cylinder 122 may extend from the top end of the stem 120 to the bottom end of the stem 120. For example, the top face of the virtual cylinder 122 may be on the same plane as at least one top surface portion of the stem 120 and the bottom face of the virtual cylinder 122 may be on the same plane as at least one bottom surface portion of the stem 120. The entire body of the stem 120 may be located within the virtual cylinder 122. That is, there is no portion of the stem 120 that is not located within the virtual cylinder 122. In some examples, a central axis of a virtual cylinder 122 of the stem 120 may coincide with the central axis 10 of the head 110. The largest distance from central axis 10 to a side surface of the stem 120 may be the radius of the virtual cylinder 122. In some examples, the virtual cylinder 122 may be defined as having i) a central axis 10 coinciding with the central axis 10 of the head 110, ii) a length extending along the central axis 10 from the top end of the stem 120 to the bottom end of the stem 120, and iii) a radius that is the same as the largest distance from the central axis 10 to a side surface of the stem 120.

In some examples, the outside surface of stem 120 that faces the vacant portion of the virtual cylinder 122 may include a smooth surface. As used herein, a smooth surface may mean a surface without sharp, angled edges. In some examples, a surface of the stem 120 facing the vacant portions of the virtual cylinder 122 includes an angled or a curved surface. The angled or a curved surface may provide a better angle of insertion for implantation of prosthesis 100.

In some examples, at least one cross-sectional area of the stem 120, perpendicular to the central axis 10, may include a width equal to the diameter of the virtual cylinder 122. This may ensure stability of the stem 120. In some examples, at least one cross-sectional area of the stem 120, perpendicular to the central axis 10, may be circular in shape and may have a circumference the same as the circumference of the virtual cylinder 122. In other examples, at least one cross-sectional area of the stem 120, perpendicular to the central axis 10, may be circular in shape and may have a circumference smaller than the circumference of the virtual cylinder 122.

In some examples, a volume ratio between the portion occupied by the stem 120 and the vacant portion in the virtual cylinder 122 may be in a range of about 1:1.5 to about 7:1, such as in a range of about 1:1.5 to about 1:1, about 2:1 to about 3:1, about 3:1 to about 4:1, about 4:1 to about 5:1, about 5:1 to about 6:1, or about 6:1 to about 7:1.

Still referring to FIG. 1A, the stem 120 may define a plane 20 orthogonal to the central axis 10. The stem may define a first axis 30, which may be on the plane 20 and may pass through the central axis 10 of the stem 120. In some examples, the first axis 30 may pass through the side surface portion of the stem 120 that coincides with the virtual cylinder 122. The stem 120 may define a second axis 40, which may be on the plane 20 and may pass through the central axis 10 of the stem 120. In some examples, the second axis 40 does not pass through the side surface portion of the stem 120 that coincides with the virtual cylinder 122.

In some examples, the first axis 30 and the second axis 40 may be orthogonal to each other, such that the first axis 30, the second axis 40, and the central axis 10 make up an x-axis, a y-axis, and a z-axis, respectively. In some examples, the first axis 30 and the second axis 40 may be at a non-90° angle relative to one another, such between a 60° angle and a 90° angle, for example, between a 60° angle and a 70° angle, between a 70° angle and an 80° angle, or between an 80° angle and a 90° angle.

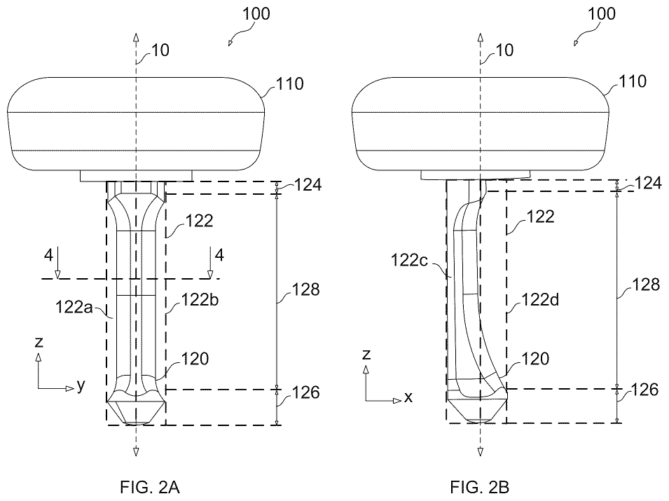

Referring to FIGS. 2A and 2B, the stem 120 may include a first end portion 124, a second end portion 126 opposite the first end portion 124, and an intermediate portion 128 between the first end portion 124 and the second end portion 126. In some examples, the first end portion 124 may make up the top 1 to 30% of the stem 120 (e.g., near the head 110), for example the top 1-5%, 5-10%, 10-15%, 15-20%, 20-25%, or 25-30% of the stem. In some examples, the second end portion 126 may make up the bottom 1 to 30% of the stem 120, for example the bottom 1-5%, 5-10%, 10-15%, 15-20%, 20-25%, or 25-30% of the stem 120.

In some examples, the plane 20 is disposed in the intermediate portion 128. In some examples, the plane 20 may be disposed at the middle of the stem 120 and the virtual cylinder 122. In other examples, the plane 20 may be disposed at any other suitable part of the intermediate portion 128.

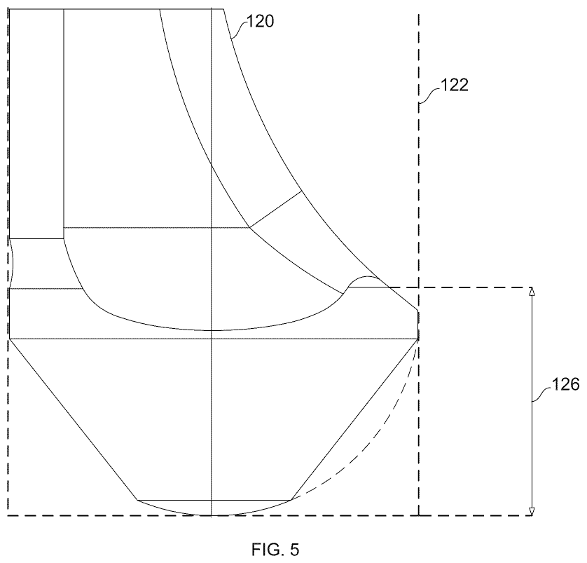

In some examples, as shown in FIG. 5, at least a portion of the second end portion 126 of the stem 120 may be curved or partially spherical with some portions removed. In some examples, a side surface of the second end portion 126 defines one or more chords of the partial sphere. Alternatively, the second end portion 126 may be spherical. A portion of the second end portion 126 may coincide with a surface of the partial sphere. For example, the bottom surface of the second end portion 126 may coincide with a surface of the partial sphere. Additionally or alternatively, the side surface of the second end portion 126 may coincide with a portion of the surface of the partial sphere.

The second end portion 126 of the stem 120 may include a first end coupled to the intermediate portion 128 and a second end opposite the first end. At least a portion of the second end portion 126 may include the second end and a largest width of the portion may taper toward the second end.

FIG. 2A illustrates the prosthesis 100 when viewed from the first axis 30 (i.e. first axis 30 going into the page). As shown, in this 2-dimensional view, a portion of the virtual cylinder 122 may be covered by the shaft of the stem 120 and a portion of the virtual cylinder 122 may be vacant. From this 2-dimensional view, the virtual cylinder 122 may be divided, by the central axis 10, into a first portion 122a (on the left side of the central axis 10) and a second portion 122b (on the right side of the central axis 10).

In some examples, when viewed from the first axis 30, the stem 120 covers over 80% (e.g., 80-85%, 85-90%, 90-95% or 95-100%) of the first portion 122a. In some examples, when viewed from the first axis 30, the vacant portion covers under 20% (e.g., 0-5%, 5-10%, 10-15%, or 15-20%) of the first portion 122a. In some examples, when viewed from the first axis 30, the stem 120 covers over 80% (e.g., 80-85%, 85-90%, 90-95% or 95-100%) of the second portion 122b. In some examples, when viewed from the first axis 30, the vacant portion covers under 20% (e.g., 0-5%, 5-10%, 10-15%, or 15-20%) of the second portion 122b.

In some examples, when viewed from the first axis 30, the stem 120 may be symmetrical about the central axis 10. In other examples, when viewed from the first axis 30, the stem 120 may be asymmetrical about the central axis 10.

In some examples, when viewed from the first axis 30, the largest width of the first end portion 124 and/or the second end portion 126 may be greater than the smallest width of the intermediate portion 128. Additionally or alternatively, when viewed from the first axis 30, the largest width of the first end portion 124 may be the same as the width of the virtual cylinder 122. In some examples, when viewed from the first axis 30, the largest width of the second end portion 126 may be the same as the width of the virtual cylinder 122. In some examples, the smallest width of the intermediate portion 128 may taper to a larger width of the first end portion 124 and/or the second end portion 126. As used herein, a width of a portion of the stem 120 when viewed from an axis (e.g., first axis) may refer to a distance, in a 2-dimensional view from the axis (e.g., first axis), between two points of the stem 120 that are i) on a side surface of the stem 120 and ii) on a plane perpendicular to the central axis 10.

FIG. 2B illustrates the prosthesis 100 when viewed from the second axis 40 (i.e. second axis 40 going into the page). As shown, a portion of the virtual cylinder 122 may be covered by the shaft of the stem 120 and a portion of the virtual cylinder 122 may be vacant. From this 2-dimensional view, the virtual cylinder 122 may be divided, by the central axis 10, into a third portion 122c (on the left side of the central axis 10) and a fourth portion 122d (on the right side of the central axis 10). In some examples, when viewed from the second axis 40, the stem 120 covers over 80%, 90%, or 95% (e.g. 85%, 87%, 92%, 96%, 97%, 98%, 99%, or 100%) of the third portion 122c. In some examples, when viewed from the second axis 40, less than 90%, 50%, or 30% (10-20%, 20-30%, 30-40%, 40-50%, 50-60%, 60-70%, 70-80%, 80-90%) of the fourth portion 122d may be covered by the stem 120.

In some examples, the first axis 30 may be defined as an axis on the plane 20, where, in a 2-dimensional view from this axis, the percentage of the portion of the virtual cylinder 122 that is covered by the stem 120 (hereinafter “stem coverage percentage” of the virtual cylinder 122) is maximum (e.g., over 70%, 80% or 90%) compared to a 2-dimensional view from other axes on the plane 20. That is, in this 2-dimensional view from the first axis 30, the least portion of the virtual cylinder 122 is covered by the vacant portion compared to a 2-dimensional view from other axes on the plane 20.

In some examples, the second axis 40 may be defined as an axis on the plane 20, where, in a 2-dimensional view from this axis, the stem coverage percentage of the virtual cylinder 122 is minimum (e.g., less than 90%, 80%, 70% or 60%) compared to a 2-dimensional view from other axes on the plane 20. That is, in this 2-dimensional view from the second axis 40, the largest portion of the virtual cylinder 122 is covered by the vacant portion (compared to a 2-dimensional view from other axes on the plane 20).

In some examples, in a 2-dimensional view from the second axis 40, the difference between the stem coverage percentage of the third portion 122c and the stem coverage percentage of the fourth portion 122d is maximum compared to a 2-dimensional view from other axes on the plane 20. In some examples, in a 2-dimensional view from the second axis 40, a ratio between the stem coverage percentage of the third portion 122c and the stem coverage percentage of the fourth portion 122d is in a range of about 10:1 to about 1.2:1, for example, about 10:1 to about 8:1, about 8:1 to about 6:1, about 6:1 to about 4:1, about 4:1 to about 2:1, or about 2:1 to about 1.2:1.

In some examples, as depicted in FIG. 2B, the central axis 10 may transverse at least a portion of the vacant portion of the virtual cylinder 122. Stated differently, in some examples, the smallest width of the stem 120 of the virtual cylinder 122 (when viewed from the first axis 30 or the second axis 40) may be smaller than a radius of the virtual cylinder 122. In other examples, the central axis 10 may only traverse the stem 120 portion of the virtual cylinder 122 and the smallest width of the stem 120 within the virtual cylinder 122 (when viewed from the first axis 30 or the second axis 40) may be larger than a radius of the virtual cylinder 122.

When viewed from the second axis 40, the smallest width of the first end portion 124 may be greater that the smallest width of the intermediate portion 128. Additionally or alternatively, when viewed from the second axis 40, the largest width of the second end portion 126 may be greater than the smallest width of the intermediate portion 128.

In some examples, when viewed from the second axis 40, the largest width of the first end portion 124 may be smaller than the diameter of the virtual cylinder 122. In other examples, when viewed from the second axis 40, the largest width of the first end portion 124 may be the same as the diameter of the virtual cylinder 122.

In some examples, when viewed from the second axis 40, the largest width of the second end portion 126 may be the same as the diameter of the virtual cylinder 122. In other examples, when viewed from the second axis 40, the largest width of the second end portion 126 may be smaller than the diameter of the virtual cylinder 122. In some examples, the smallest width of the intermediate portion 128 may taper to a larger width of the first end portion 124 and/or the second end portion 126.

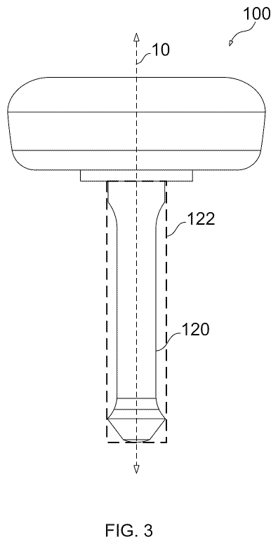

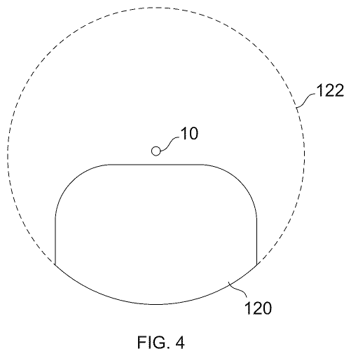

FIG. 3 illustrates prosthesis 100 when viewed from the first axis 30 from the direction opposite from FIG. 2A. FIG. 4 illustrates a cross-sectional view taken along the line 4-4 of FIG. 2A. As shown in FIGS. 2B, 3, and 4, a side surface portion of the stem 120 may coincide with a surface of the virtual cylinder 122.

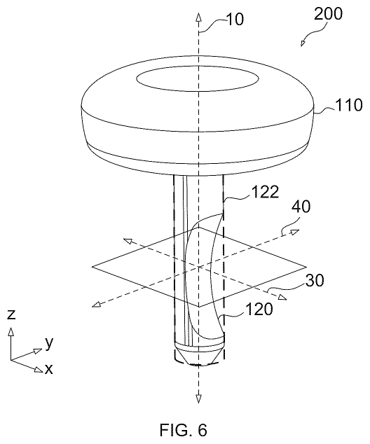

FIGS. 6 to 10 illustrate an example prosthesis 200 according to another example of the present disclosure. The prosthesis 200 may be similar to the prosthesis 100, but with different stem dimensions and/or shapes. Various configurations/features/characteristics of the prosthesis 200 (e.g., material, virtual cylinder, central axis, stem coverage percentage, first axis, second axis, first end portion, second end portion, intermediate portion, etc.) may be similar to and/or same as the ones described above with respect to the prosthesis 100 and, thus, duplicate description may be omitted.

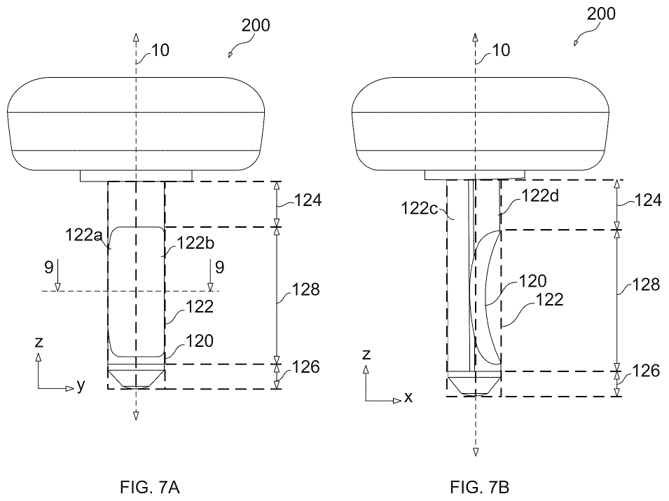

FIGS. 7A and 7B illustrate the prosthesis 200 from the first axis 30 and from the second axis 40, respectively. As shown in FIGS. 7A and 7B, the first end portion 124 may have the same width of the virtual cylinder 122 when viewed from both the first axis 30 and the second axis 40. The first end portion 124 of stem 120 may occupy the whole volume of the virtual cylinder 122, while the intermediate portion 128 may include one or more vacant portions of the virtual cylinder 122. In some examples, when viewed from the first axis 30, the intermediate portion 128 may have the same width as the virtual cylinder 122.

As shown in FIG. 7B, when viewed from the second axis 40, in some examples, the stem 120 may cover over 90%, for example, over 90%, 95% or 99%, or cover 100% of the third portion 122c of the virtual cylinder 122. In some examples, when viewed from the second axis 40, the stem 120 may cover between 30-85%, for example between about 30-40%, about 40-50%, about 50-60%, about 60-70%, about 70-80%, or about 80-85% of the fourth portion 122d of the virtual cylinder 122.

While FIG. 7B shows the first end portion 124 and the intermediate portion 128 of the stem 120 covering 100% of the third portion 122c of the virtual cylinder 122 when viewed from the second axis 40, it can be appreciated that the first end portion 124 and the intermediate portion 128 of the stem 120 may cover less than 100% of the third portion 122c of the stem 120.

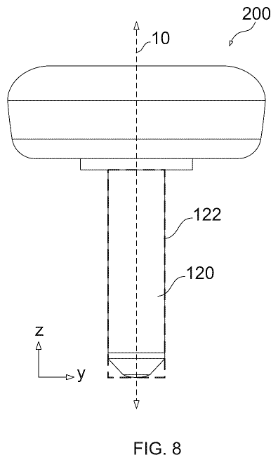

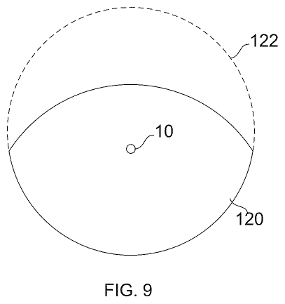

FIG. 8 illustrates prosthesis 200 when viewed from the first axis 30 from the direction opposite from FIG. 7A. As shown in FIG. 9, when viewed from the first axis 30 from an opposite direction, the first end portion and the intermediate portion of the stem 120 cover 100% of the virtual cylinder 122. From this view, the entire surface of the first end portion and the intermediate portion of the stem 120 coincides with a portion of the surface of the virtual cylinder 122.

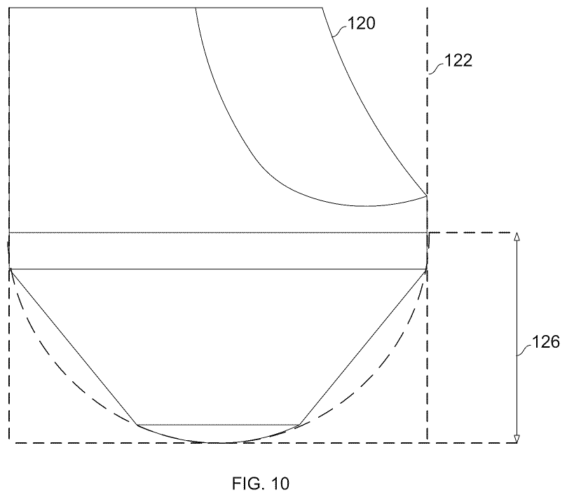

FIG. 10 illustrates an example of the second end portion 126 of the stem 120 of the prosthesis 200. At least a portion of the second end portion 126 of the stem 120 may be curved or partially spherical with some portions removed. In some examples, a side surface of the second end portion 126 defines one or more chords of the partial sphere. Alternatively, the second end portion 126 may be spherical. A portion of the second end portion 126 may coincide with a surface of the partial sphere. For example, the bottom surface of the second end portion 126 may coincide with a surface of the partial sphere.

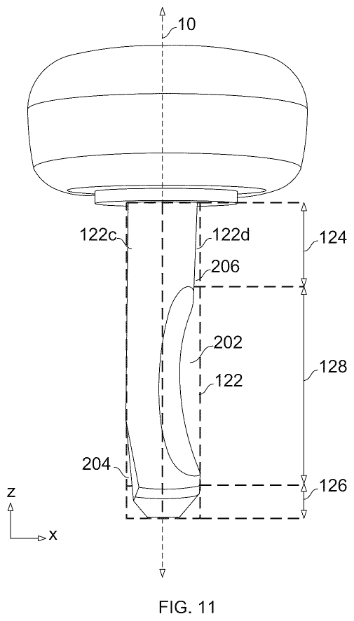

FIGS. 11 to 13 illustrate modified prostheses of FIG. 6. As depicted in FIG. 11, in some examples, portions of the stem 120 may be removed from the first end portion 124, intermediate portion 128, and/or second end portion 126 of the stem 120. In some examples, the intermediate portion 128 of the stem may include a primary, first vacant portion 202. Additionally, a second vacant portion 204 may be formed in the lower part of the intermediate portion 128 and a portion of the second end portion 126. A third vacant portion 206 may be formed in the first end portion 124. In some examples, the second and/or third vacant portions 204, 206 may taper along the length.

The second vacant portion 204 may be opposite the first vacant portion 202 and/or the third vacant portion 206. For example, when viewed from the second axis 40 as depicted in FIG. 11, the first vacant portion 202 and/or the third vacant portion 206 may be formed in the fourth portion 122d contacting the right side of the virtual cylinder 122, while the second vacant portion 204 may be formed in the third portion 122c contacting the left side of the virtual cylinder 122. This configuration may limit the surface area of the stem 120 that has to inserted into the radial canal at an angle.

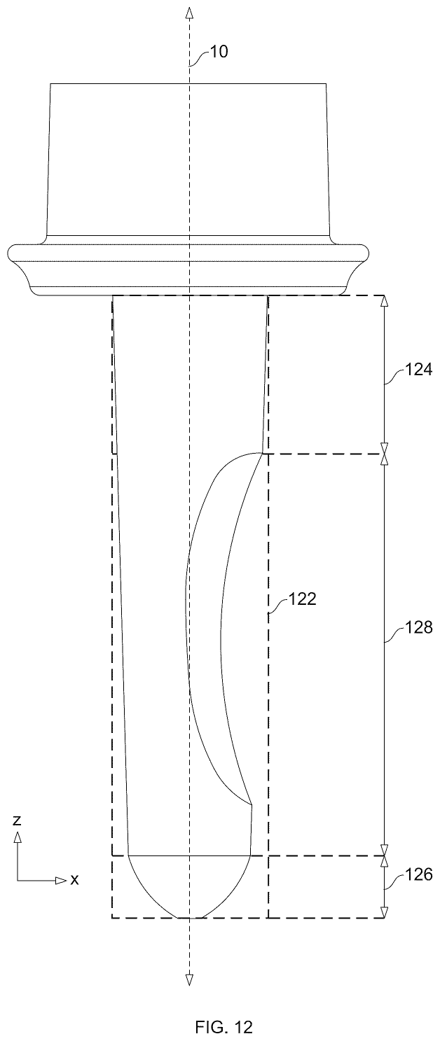

As depicted in FIGS. 12 and 13, in some examples, the stem 120 may taper from a largest width or diameter in at least a portion of first end portion 124 to a smaller width or diameter towards the second end portion 126. Stated differently, in some examples, the largest width of the first end portion 124 may be greater than a largest width of the second end portion 126 and/or the largest width of the intermediate portion 128.

FIG. 14 illustrates a transparent prosthesis 300 of FIG. 12 overlaid on the prosthesis 400 of FIG. 13. As shown, the prosthesis 400 depicted in FIG. 13 has slightly more volume removed in the intermediate portion 128 of the stem 120 from virtual cylinder 122 than the prosthesis 300 shown in FIG. 12. More portions removed as depicted in FIG. 13, in portions that typically interfere with the radial canal, allows better ease of insertion.

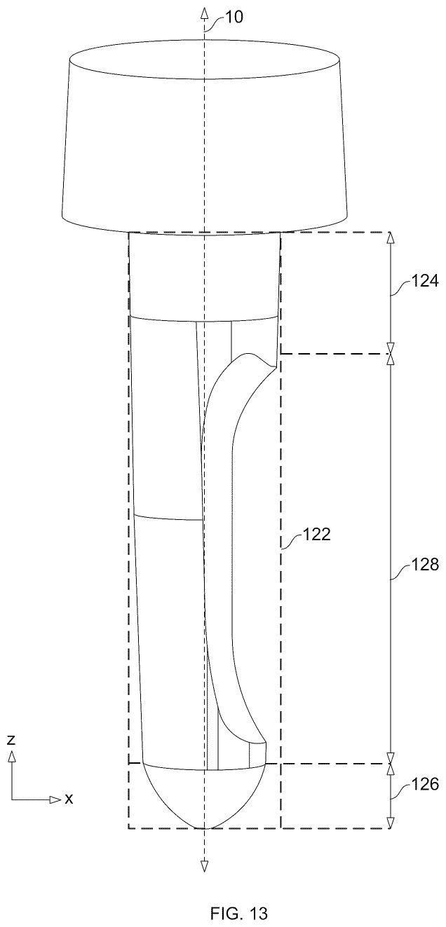

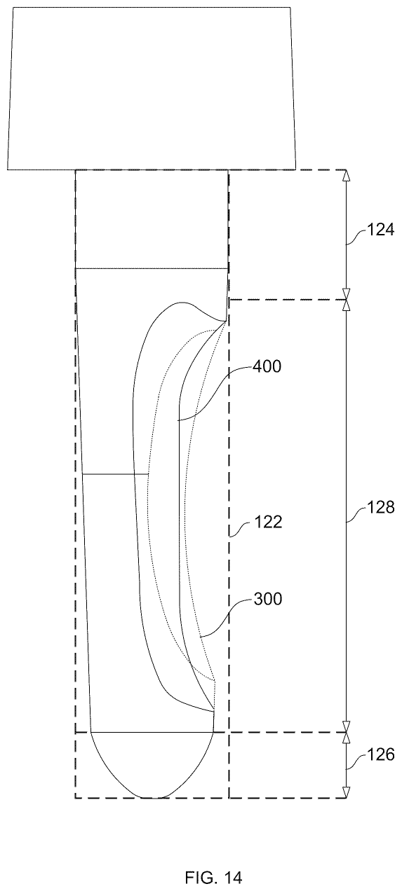

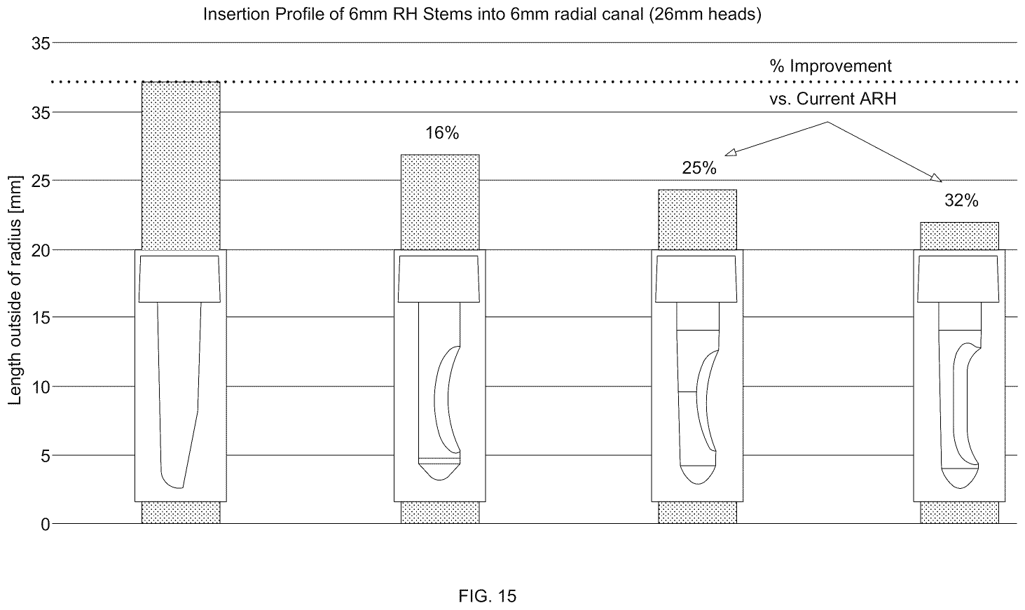

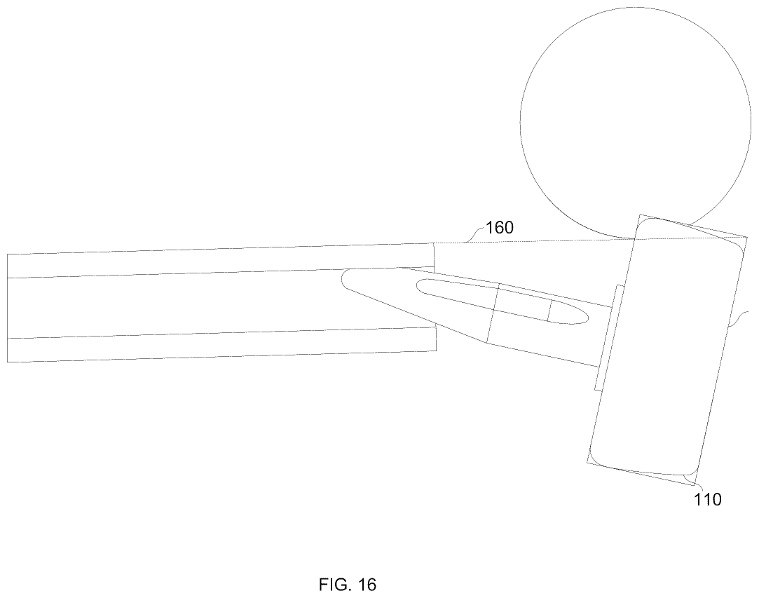

An easier insertion refers to the ability of the stem to be inserted into the radial canal at a greater depth without interference of the head with the capitellum. FIG. 15 illustrates a graph depicting a relative comparison of ease of insertion according to an example of the present disclosure measured for different embodiments of the radial head prosthesis. In some examples, ease of insertion may be measured using length outside of radius (LOR) 160 as depicted in FIG. 16. For purposes of measuring the LOR, it is assumed that the head 110 has a largest height of 11 mm and a largest width/diameter of 26 mm (for example, at least when viewed from the second axis). The LOR may be measured from the edge of the radius to a point representing the intersection of the side and top of the minimum bounding box 111 of the head 110.

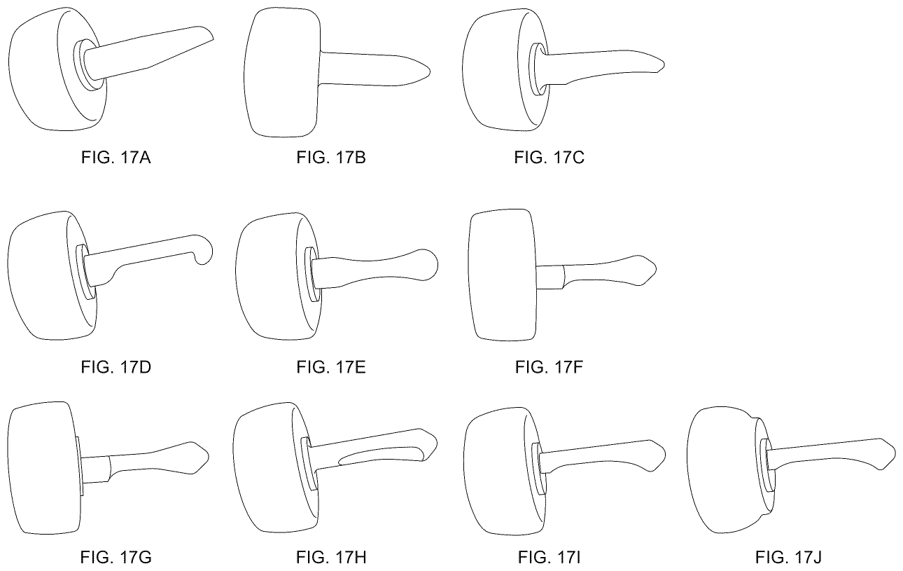

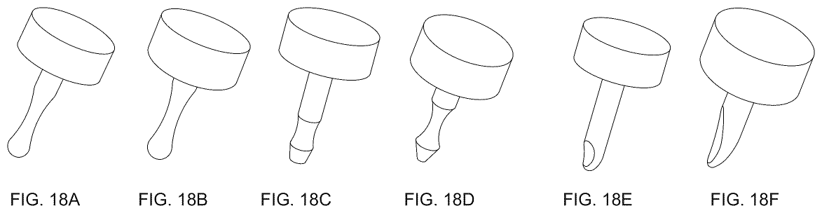

While the geometries of radial head prosthesis 100 and 200 are shown in FIGS. 1 through 13, respectively, it can be appreciated that other geometries of radial head prostheses are suitable. FIGS. 17A-17J and 18A-18F illustrate other example embodiments of a radial head prosthesis according to examples of the present disclosure.

### Embodiments

Various aspects of the subject matter described herein are set out in the following numbered embodiments:

Embodiment 1. A prosthesis for replacement of a proximal end of a radius bone comprising: a head; and a stem defining a virtual cylindrical shaft having a central axis, wherein the stem includes a side surface portion coinciding with a side surface portion of the virtual cylindrical shaft, wherein the virtual cylindrical shaft comprises: a portion occupied by the stem; and a vacant portion, wherein the stem further defines: a plane perpendicular to the central axis of the virtual cylindrical shaft; a first axis on the plane and passing through the central axis of the virtual cylindrical shaft; and a second axis on the plane, passing through the central axis of the virtual cylindrical shaft, and perpendicular to the first axis, wherein, when viewed from the first axis: the virtual cylindrical shaft is divided, by the central axis, into a first portion and a second portion, and over 80% of the first and second portions are covered by the stem; and wherein, when viewed from the second axis: the virtual cylindrical shaft is divided, by the central axis, into a third portion and a fourth portion, and over 95% of the third portion is covered by the stem and less than 90% of the fourth portion is covered by the stem.

Embodiment 2. The prosthesis of embodiment 1, wherein, when viewed from the second axis, less than 50% of the fourth portion is covered by the stem.

Embodiment 3. The prosthesis of embodiment 2, wherein, when viewed from the second axis, less than 30% of the fourth portion is covered by the stem.

Embodiment 4. The prosthesis of any one of embodiments 1-3, wherein, when viewed from the second axis, over 97% of the third portion is covered by the stem.

Embodiment 5. The prosthesis of any one of embodiments 1-4, wherein a volume ratio between the portion occupied by the stem and the vacant portion in the virtual cylindrical shaft is in a range of about 1:1.5 to about 7:1.

Embodiment 6. The prosthesis of embodiment 5, wherein a volume ratio between the portion occupied by the stem and the vacant portion in the virtual cylindrical shaft is in a range of about 1:1.5 to about 1.5:1.

Embodiment 7. The prosthesis of any one of embodiments 1-6, wherein the first axis passes through the side surface portion of the stem, and the second axis does not pass through the side surface portion of the stem.

Embodiment 8. The prosthesis of any one of embodiments 1-7, wherein at least one cross-sectional area of the stem has a circular shape having a circumference the same as a circumference of the virtual cylindrical shaft, wherein the at least one cross-sectional area of the stem is perpendicular to the central axis of the virtual cylindrical shaft.

Embodiment 9. The prosthesis of any one of embodiments 1-8, wherein the stem comprises: a first end portion; a second end portion opposite the first end portion; and an intermediate portion disposed between the first end portion and the second end portion, wherein, when viewed from the second axis, a smallest width of the first end portion is greater than a smallest width of the intermediate portion.

Embodiment 10. The prosthesis of embodiment 9, wherein, when viewed from the second axis, a largest width of the second end portion of the stem is greater than a smallest width of the intermediate portion.

Embodiment 11. The prosthesis any one of embodiments 9 and 10, wherein, when viewed from the second axis, a largest width of the first end portion of the stem is less than a width of the virtual cylindrical shaft.

Embodiment 12. The prosthesis of any one of embodiments 9-11, wherein, when viewed from the second axis, a largest width of the second end portion of the stem is the same as a width of the virtual cylindrical shaft.

Embodiment 13. The prosthesis of any one of embodiments 9-12, wherein, when viewed from the second axis, a width of the stem tapers from a smaller width in the intermediate portion to a larger width in the first end portion and/or the second end portion.

Embodiment 14. The prosthesis of any one of embodiments 9-13, when viewed from the first axis, a largest width of the first end portion is greater than a smallest width of the intermediate portion.

Embodiment 15. The prosthesis of embodiment 14, wherein, when viewed from the first axis, a largest width of the first end portion is the same as a width of the virtual cylindrical shaft.

Embodiment 16. The prosthesis of any one of embodiments 9-15, wherein, when viewed from the first axis, a largest width of the second end portion is greater than a smallest width of the intermediate portion.

Embodiment 17. The prosthesis of embodiment 16, wherein, when viewed from the first axis, the largest width of the second end portion is the same as a width of the virtual cylindrical shaft.

Embodiment 18. The prosthesis of any one of embodiments 9-17, wherein the second end portion of the stem comprises a first end coupled to the intermediate portion and a second end opposite the first end, wherein at least a portion of the second end portion includes the second end and a largest width of the portion tapers toward the second end.

Embodiment 19. The prosthesis of any one of embodiments 1-18, wherein the central axis of the virtual cylindrical shaft transverses at least a portion of the vacant portion of the virtual cylindrical shaft.

Embodiment 20. The prosthesis of any one of embodiments 1-19, wherein a portion of a surface of the stem that faces the vacant portion of the virtual cylindrical shaft comprises a smooth surface.

Embodiment 21. The prosthesis of any one of embodiments 1-20, wherein a portion of a surface of the stem that faces the vacant portion of the virtual cylindrical shaft comprises an angled surface.

Embodiment 22. The prosthesis of any one of embodiments 1-21, wherein, when viewed from the first axis, the stem is symmetrical relative to the central axis of the virtual cylindrical shaft.

Embodiment 23. The prosthesis of any one of embodiments 1-22, wherein the head defines a central axis coinciding with the central axis of the virtual cylindrical shaft.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

Additionally, in describing the components of the system of the present disclosure, there may be terms used like first, second, third, and fourth. These terms may be used for the purpose of differentiating one component from the other, but not to imply or suggest the substances, order, sequence, or number of the components.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A prosthesis for replacement of a proximal end of a radius bone comprising:
a head; and
a stem defining a virtual cylindrical shaft having a central axis, wherein the stem includes a side surface portion coinciding with a side surface portion of the virtual cylindrical shaft,
wherein the virtual cylindrical shaft comprises:
a portion occupied by the stem; and
a vacant portion,
wherein the stem further defines:
a plane perpendicular to the central axis of the virtual cylindrical shaft;
a first axis on the plane and passing through the central axis of the virtual cylindrical shaft; and
a second axis on the plane, passing through the central axis of the virtual cylindrical shaft, and perpendicular to the first axis,
wherein, when viewed from the first axis:
the virtual cylindrical shaft is divided, by the central axis, into a first portion and a second portion, and over 80% of the first and second portions are covered by the stem; and
wherein, when viewed from the second axis:
the virtual cylindrical shaft is divided, by the central axis, into a third portion and a fourth portion, and over 95% of the third portion is covered by the stem and less than 90% of the fourth portion is covered by the stem.

2. The prosthesis of claim 1, wherein, when viewed from the second axis, less than 50% of the fourth portion is covered by the stem.

3. The prosthesis of claim 2, wherein, when viewed from the second axis, less than 30% of the fourth portion is covered by the stem.

4. The prosthesis of claim 1, wherein, when viewed from the second axis, over 97% of the third portion is covered by the stem.

5. The prosthesis of claim 1, wherein a volume ratio between the portion occupied by the stem and the vacant portion in the virtual cylindrical shaft is in a range of about 1:1.5 to about 7:1.

6. The prosthesis of claim 5, wherein a volume ratio between the portion occupied by the stem and the vacant portion in the virtual cylindrical shaft is in a range of about 1:1.5 to about 1.5:1.

7. The prosthesis of claim 1, wherein the first axis passes through the side surface portion of the stem, and the second axis does not pass through the side surface portion of the stem.

8. The prosthesis of claim 1, wherein at least one cross-sectional area of the stem has a circular shape having a circumference the same as a circumference of the virtual cylindrical shaft, wherein the at least one cross-sectional area of the stem is perpendicular to the central axis of the virtual cylindrical shaft.

9. The prosthesis of claim 1, wherein the stem comprises:
a first end portion;
a second end portion opposite the first end portion; and
an intermediate portion disposed between the first end portion and the second end portion,
wherein, when viewed from the second axis, a smallest width of the first end portion is greater than a smallest width of the intermediate portion.

10. The prosthesis of claim 9, wherein, when viewed from the second axis, a largest width of the second end portion of the stem is greater than a smallest width of the intermediate portion.

11. The prosthesis of claim 9, wherein, when viewed from the second axis, a largest width of the first end portion of the stem is less than a width of the virtual cylindrical shaft.

12. The prosthesis of claim 9, wherein, when viewed from the second axis, a largest width of the second end portion of the stem is the same as a width of the virtual cylindrical shaft.

13. The prosthesis of claim 9, wherein, when viewed from the second axis, a width of the stem tapers from a smaller width in the intermediate portion to a larger width in the first end portion and/or the second end portion.

14. The prosthesis of claim 9, wherein, when viewed from the first axis, a largest width of the first end portion is greater than a smallest width of the intermediate portion.

15. The prosthesis of claim 14, wherein, when viewed from the first axis, a largest width of the first end portion is the same as a width of the virtual cylindrical shaft.

16. The prosthesis of claim 9, wherein, when viewed from the first axis, a largest width of the second end portion is greater than a smallest width of the intermediate portion.

17. The prosthesis of claim 16, wherein, when viewed from the first axis, the largest width of the second end portion is the same as a width of the virtual cylindrical shaft.

18. The prosthesis of claim 9, wherein the second end portion of the stem comprises a first end coupled to the intermediate portion and a second end opposite the first end, wherein at least a portion of the second end portion includes the second end and a largest width of the portion tapers toward the second end.

19. The prosthesis of claim 1, wherein the central axis of the virtual cylindrical shaft transverses at least a portion of the vacant portion of the virtual cylindrical shaft.

20. The prosthesis of claim 1, wherein a portion of a surface of the stem that faces the vacant portion of the virtual cylindrical shaft comprises a smooth surface.

21. The prosthesis of claim 1, wherein a portion of a surface of the stem that faces the vacant portion of the virtual cylindrical shaft comprises an angled surface.

22. The prosthesis of claim 1, wherein, when viewed from the first axis, the stem is symmetrical relative to the central axis of the virtual cylindrical shaft.

23. The prosthesis of claim 1, wherein the head defines a central axis coinciding with the central axis of the virtual cylindrical shaft.
